(19) United States
(12) Patent Application Publication
(10) Pub. No.: US 2007/0026511 A1
(43) Pub. Date: Feb. 1, 2007

(54) METHODS FOR THE ADMINISTRATION OF FV AND RELATED COMPOSITIONS

(76) Inventors: Edward Stephen Morrissey, Ojai, CA (US); Ruotao Wang, Beijing (CN)

Correspondence Address: SHEPPARD, MULLIN, RICHTER & HAMPTON LLP, 333 SOUTH HOPE STREET, 48TH FLOOR, LOS ANGELES, CA, 90071-1448, US

(21) Appl. No.: 11/457,376

(22) Filed: Jul. 13, 2006

Related U.S. Application Data
(60) Provisional application No. 60/699,320, filed on Jul. 13, 2005.

(51) Int. Cl. C12N 9/04

(52) U.S. Cl. 435/190

(57) ABSTRACT

The present invention is generally directed to methods for the administration of compositions related to improved physical performance and post-exertion recovery, as well as the administered compositions. It is more specifically directed to compositions containing a FV extract and methods for using such compositions that, among other things, improve lactic acid clearance, muscle reaction time, stamina, exertion recovery time and reduce fatigue. In an extract aspect, the Flammulina velutipes extract has the following characteristics: a moisture content ranging from 0 percent to 10 percent; a protein content ranging from 20 percent to 40 percent; a carbohydrate content ranging from 40 percent to 80 percent; an ash content ranging from 0 percent to 15 percent; and, a fat content ranging from 0 percent to 5 percent.

### CROSS-REFERENCE TO RELATED APPLICATION

This application claims priority to U.S. Provisional Patent Application Ser. No. 60/699,320 filed on Jul. 13, 2005, the entire disclosure of which is incorporated by reference.

### FIELD OF THE INVENTION

The present invention is generally directed to methods for the administration of compositions related to improved physical performance and post-exertion recovery, as well as the administered compositions. It is more specifically directed to compositions containing a FV (i.e., Flammulina velutipes) extract and methods for using such compositions that, among other things, improve lactic acid clearance, muscle reaction time, stamina, exertion recovery time and reduce fatigue.

### BACKGROUND OF THE INVENTION

A product with the name “2^{nd} Wind” was sold briefly as an athletic recovery formula. The product included six powdered ingredients, some of which were based on three types of non-extracted, powdered mushrooms. The FY mycelium accounted for 50% of the formula. The six ingredients were as follows: FV; Eleuthero root; Reishi; Citrus peel; Ginseng root; and, Cordyceps sinensis.

The effect of 2^{nd} Wind was studied in mice. Groups of mice were fed 2^{nd} Wind or placebo for 21 days. After 21 days, the mice performed a loaded swim test to exhaustion. Total swim time was recorded. Blood samples were taken before and immediately after the swim, as well as 5 and 10 minutes post-swim, and were analyzed for lactate content. After 10 minutes of rest, the mice performed an unloaded swim test to exhaustion; the total swim time was recorded.

Compared to control, 2nd Wind was observed to significantly decrease peak lactate levels after swimming and to significantly increase both the initial swim time and the post-recovery swim time. In particular, 2^{nd} Wind (a) reduced peak lactate levels by 41% compared to control; (b) reduced the increase in peak blood lactate levels compared to rest: the 2^{nd} Wind group experienced only a 68% increase compared to rest, while the control group increased by 174%; (c) reduced blood lactate levels after a 10-minute recovery compared to rest: the 2^{nd} wind group showed only a 22% increase compared to rest, while the control group showed a 135% increase for the control group; (d) increased swim time by 93% compared to control; and (e) increased post-recovery swim time by 179% compared to control.

### SUMMARY OF THE INVENTION

The present invention is generally directed to methods for the administration of compositions related to improved, physical performance, as well as the administered compositions. It is more specifically directed to compositions containing a FV extract and methods for using such compositions that, among other things, improve lactic acid clearance, muscle reaction time, stamina, exertion recovery time and reduce fatigue.

In an extract aspect, the Flammulina velutipes extract has the following characteristics: a moisture content ranging from 0 percent to 10 percent; a protein content ranging from 20 percent to 40 percent; a carbohydrate content ranging from 40 percent to 80 percent; an ash content ranging from 0 percent to 15 percent; and, a fat content ranging from 0 percent to 5 percent.

In certain cases, the extract is made using a method comprising the following steps: comminution of a Flammulina velutipes material to form a powder; extraction of the powder using a solvent to provide a mixture of soluble and insoluble components; separating the soluble component from the insoluble component; concentrating the soluble component to provide a concentrate; and, drying the concentrate.

In certain cases, the Flammulina velutipes material comprises one or more Flammulina velutipes fruiting bodies.

In other cases, the Flammulina velutipes material comprises fermented Flammulina velutipes mycelium.

In other cases, the Flammulina velutipes material comprises Flammulina velutipes fermentation medium comprising biochemical fermentation products.

In other cases, the Flammulina velutipes material consists essentially of one or more Flammulina velutipes fruiting bodies.

In other cases, the Flammulina velutipes material consists essentially of fermented Flammulina velutipes mycelium.

In other cases, the Flammulina velutipes material consists essentially of Flammulina velutipes fermentation medium comprising biochemical fermentation products.

In other cases, the Flammulina velutipes material consists essentially of Flammulina velutipes mycelium.

In other cases, the Flammulina velutipes material consists essentially of Flammulina velutipes spores.

In a composition aspect, the composition includes a Flammulina velutipes extract and is formulated into a delivery form selected from a list consisting of capsules, tablets, a powdered drink mix, a ready-to-drink beverage, a semisolid, a food, and a supplement bar.

In certain cases, the extract is a powder having the following characteristics: a moisture content ranging from 0 percent to 10 percent; a protein content ranging from 20 percent to 40 percent; a carbohydrate content ranging from 40 percent to 80 percent; an ash content ranging from 0 percent to 15 percent; and, a fat content ranging from 0 percent to 5 percent.

In certain cases, the extract is formulated into a capsule, and wherein the formulated material further comprises at least one ingredient from the following list: flour; starch; modified starch; maltodextrin; cellulose; modified cellulose; protein hydrolysate; rice powder; whey powder; calcium phosphate; calcium carbonate; lactose; saccharides; sorbitol; mannitol; xylitol; stearic acid; stearate; silica; silicate; polyethylene glycol; flavors; and, colors.

In other cases, the extract is formulated into a tablet, and wherein the formulated material further comprises at least one ingredient from the following list: starch; modified starch; maltodextrin; cellulose; methylcellulose; ethylcellulose; hydroxypropylmethylcellulose; modified cellulose; protein hydrolysate; rice powder; whey powder; calcium phosphate; calcium carbonate; lactose; sweeteners; sorbitol; mannitol; xylitol; zein; saccharides; stearic acid; stearate; silica; silicate; polyethylene glycol; pharmaceutical glaze; wax; flavors; and colors.

In other cases, the extract is formulated into a powdered drink mix, and wherein the formulated material further comprises at least one ingredient from the following list: starch; modified starch; maltodextrin; cellulose; modified cellulose; protein hydrolysate; whey powder; calcium phosphate; calcium carbonate; lactose; sorbitol; mannitol; xylitol; sweeteners; stearic acid; stearate; silica; silicate; flavors; and, colors.

In other cases, the extract is formulated into a ready-to-drink beverage, and wherein the formulated material further comprises at least one ingredient from the following list: starch; modified starch; maltodextrin; cellulose; modified cellulose; protein hydrolysate; whey powder; calcium phosphate; calcium carbonate; lecithin; sweeteners; sorbitol; mannitol; xylitol; silica; silicate; solvents; acidifiers; citrate; preservatives; caffeine; flavors; and, colors.

In other cases, the extract is formulated into a semisolid, and wherein the formulated material further comprises at least one ingredient from the following list: starch; modified starch; maltodextrin; cellulose; modified cellulose; protein hydrolysate; whey powder; calcium phosphate; calcium carbonate; lecithin oil; partially hydrogenated oil; fat; milk; milk solids; mono- or diglycerides; polysorbates; sorbitan monostearate; sweeteners; sorbitol; mannitol; xylitol; silica; silicate; solvents; acidifiers; citrate; preservatives; flavors; and, colors.

In other cases, the extract is formulated into a food or supplement bar, and wherein the formulated material further comprises at least one ingredient from the following list: flour; starch; modified starch; maltodextrin; cellulose; methylcellulose; ethylcellulose; hydroxypropylmethylcellulose; modified cellulose; protein hydrolysate; whey powder; calcium phosphate; calcium carbonate; lecithin; mono- or diglycerides; polysorbates; sorbitan monostearate binders; sweeteners; sorbitol; mannitol; xylitol; silica; silicate; solvents; acidifiers; citrate; preservatives; flavors; and, colors.

In a kit aspect, the kit is for improving lactic acid clearance, muscle reaction time, stamina, exertion recovery time, or reducing fatigue, and the kit comprises: a composition comprising a Flammulina velutipes extract; a container including the composition; and, instructions on how to use the composition to improve lactic acid clearance, muscle reaction time, stamina, exertion recovery time or to reduce fatigue.

In certain cases, the composition is in the form of a capsule.

In other cases, the composition is in the form of a tablet.

In other cases, the composition is in the form of a powdered drink mix.

In other cases, the composition is in the form of a ready-to-drink beverage.

In other cases, the composition is in the form of a semisolid.

In other cases, the composition is in the form of a food or supplement bar.

In a method aspect, the method provides for increasing physical stamina in a mammal and comprises administering a composition comprising a Flammulina velutipes extract to the mammal.

In certain cases, the composition is in the form of a capsule.

In other cases, the composition is in the form of a tablet.

In other cases, the composition is in the form of a powdered drink mix that has been mixed with a solvent.

In other cases, the composition is in the form of a ready-to-drink beverage.

In other cases, the composition is in the form of a semisolid.

In other cases, the composition is in the form of a food or supplement bar.

### DETAILED DESCRIPTION OF THE INVENTION

The FV extract, when dried to a moisture content of 0-10% and ground to a powder, is generally a brown powder containing approximately 20-40% protein, 40-80% carbohydrates, 0-15% ash, and 0-5% fat.

The FV extract can be used either alone or with additional physiologically active ingredients such as vitamins, minerals, amino acids, protein powders, herbs, extracts, and other nutraceuticals. The FV extract and any additional physiologically active ingredients can be formulated into various delivery systems either alone or with the aid of excipients; some examples of delivery forms and possible excipients are:
Into capsules with the addition of flour, starch, modified starch, maltodextrin, cellulose, modified cellulose, protein hydrolysate, rice powder, whey powder, calcium phosphate, calcium carbonate, lactose, saccharides, sorbitol, m-nannitol, xylitol, stearic acid, stearate, silica, silicate, polyethylene glycol, flavors, and/or colors, among others
Into tablets with the addition of starch, modified starch, maltodextrin, cellulose, methylcellulose, ethylcellulose, hydroxypropylmethylcellulose, modified cellulose, protein hydrolysate, rice powder, whey powder, calcium phosphate, calcium carbonate, lactose, sweeteners (e.g. sucrose, fructose, glucose, corn syrup, saccharides, saccharine, sucralose, aspartame, etc.), sorbitol, mannitol, xylitol, (gum tragacanth, gum arabic, agar, guar gum, locust bean gum, karaya gum, xanthan gum, etc.) zein, saccharides, stearic acid, stearate, silica, silicate, polyethylene glycol, pharmaceutical glaze, wax, flavors, and/or colors, among others.
Into powdered drink mix with the addition of starch, modified starch, maltodextrin, cellulose, modified cellulose, protein hydrolysate, whey powder, calcium phosphate, calcium carbonate, lactose, sorbitol, mannitol, xylitol, sweeteners (e.g. sucrose, fructose, glucose, corn syrup, saccharides, saccharine, sucralose, aspartame, etc.), stearic acid, stearate, silica, silicate, flavors, and/or colors, among others.
Into ready-to-drink beverages with the addition of starch, modified starch, maltodextrin, cellulose, modified cellulose, protein hydrolysate, whey powder, calcium phosphate, calcium carbonate, lecithin, sweeteners (e.g. sucrose, fructose, glucose, corn syrup, saccharides, saccharine, sucralose, aspartame, etc.), sorbitol, mannitol, xylitol, silica, silicate, solvents (e.g. water, ethanol, polyethylene glycol, propylene glycol, glycerin), acidifiers (e.g. citric acid, acetic acid, malic acid, tartaric acid), citrate, preservatives (e.g. benzoic acid, benzoate, sorbic acid, sorbate, polysorbate, propionic acid, propionate, nisin), caffeine, flavors, and/or colors, among others.
Into semisolids such as gu with the addition of starch, modified starch, maltodextrin, cellulose, modified cellulose, protein hydrolysate, whey powder, calcium phosphate, calcium carbonate, lecithin, oil, partially hydrogenated oil, fat, milk, milk solids, mono- or diglycerides, polysorbates, sorbitan monostearate, (gum tragacanth, gum arabic, agar, guar gum, locust bean gum, karaya gum, xanthan gum, etch), sweeteners (e.g. sucrose, fructose, glucose, corn syrup, saccharides, saccharine, sucralose, aspartame, etc.), sorbitol, mannitol, xylitol, silica, silicate, solvents (e.g. water, ethanol, polyethylene glycol, propylene glycol, glycerin), acidifiers (e.g. citric acid, acetic acid, malic acid, tartaric acid), citrate, preservatives (e.g. benzoic acid, benzoate, sorbic acid, sorbate, polysorbate, propionic acid, propionate, nisin, parabens), flavors, and/or colors, among others.
Into food or supplement bars with the addition of flour, starch, modified starch, maltodextrin, cellulose, methylcellulose, ethylcellulose, hydroxypropyl-methylcellulose, modified cellulose, protein hydrolysate, whey powder, calcium phosphate, calcium carbonate, lecithin, mono- or diglycerides, polysorbates, sorbitan monostearate, binders (gum tragacanth, gum arabic, agar, guar gum, locust bean gum, karaya gum, xanthan gum, etc.), sweeteners (e.g. sucrose, fructose, glucose, corn syrup, saccharides, saccharine, sucralose, aspartame, etc.), sorbitol, mannitol, xylitol, silica, silicate, solvents (e.g. water, ethanol, polyethylene glycol, propylene glycol, glycerin), acidifiers (e.g. citric acid, acetic acid, malic acid, tartaric acid), citrate, preservatives (e.g. benzoic acid, benzoate, sorbic acid, sorbate, polysorbate, propionic acid, propionate, nisin, BRHA, BHT, EDTA, TBHQ, etc.), flavors, and/or colors, among others.

The various delivery systems can be packaged in a number of ways as appropriate, including but not limited to: a bottle with a label and/or insert having instructions; a foil laminate pouch with instructions; a wrapper with instructions; and, a carton or box with instructions and/or label with instructions and/or insert with instructions.

The FV extract is made by comminution to a powder (if necessary); extraction in water, a lower alcohol, or a combination thereof; concentration, drying, and packaging. Other steps may also be included, such as precipitation of the ethanol-insoluble fraction; granulating and/or milling of the finished extract powder

The FV extract can be made from a variety of starting materials, including FV fruiting body, fermented mycelium, and/or fermentation medium containing biochemical fermentation products, and can be in dry or fresh form.

### EXAMPLES

The following examples are presented by way of illustration, not by way of limitation.

### Example 1

FV Mycelium spores are prepared and placed in a media comprised of vegetable matter (such as grain, tomatoes, soybeans, root vegetables and greens), glucose, potassium hydrogen phosphate, Magnesium sulfate, amino acids, minerals (e.g., selenium), vitamins (e.g., B1) and water and kept at a target pH of 6.9-7.1, at a temperature of 25 Celsius. 125,000 cc of the mycelium is then fermented for 100-110 hours in a media consisting of glucose, Potassium hydrogen phosphate, magnesium sulfate, amino acids, lipids and hot water. The fermentation liquid is kept at a pH of 6.9-7.1, a temperature of 25 Celsius. The mycclium is then separated from the mixture and vacuum dried. The result is milled into a powder. The powdered FV mycelium is extracted with 12 volumes of water under reflux for 2 hours. The extraction is repeated with 10 volumes of water under reflux for 2 hours. The miscella is separated from the marc by centrifugation. The miscella is concentrated in a vacuum membrane evaporator, then dried in a vacuum oven and milled to a powder.

### Example 2

FV Mycelium spores are prepared and placed in a media comprised of vegetable matter (such as grain, tomatoes, soybeans, root vegetables and greens), glucose, potassium hydrogen phosphate, Magnesium sulfate, amino acids, minerals (e.g. selenium), vitamins (e.g. B1) and water and kept at a target pH of 6.0-7.4, at a temperature of 22 Celsius. 125,000 cc of the mycelium is then fermented for 80-100 hours in a media consisting of glucose, Potassium hydrogen phosphate, magnesium sulfate, amino acids, lipids and hot water. The fermentation liquid is kept at a pH of 6.0-7.4, a temperature of 22 Celsius. The mycelium is then separated from the mixture and vacuum dried. The result is milled into a powder. The powdered FV mycelium is extracted with 12 volumes of 25% alcohol under reflux for 2 hours. The extraction is repeated with 10 volumes of 25% alcohol under reflux for 2 hours. The miscella is separated from the marc by centrifugation. The miscella is concentrated in a vacuum membrane evaporator, then dried in a vacuum oven and milled to a powder.

### Example 3

An extract of FV was tested in a placebo-controlled study. Study groups of mice swam to exhaustion, and then were tested for serum urea levels. The study results show that extracted FV reduced serum urea by 18% compared to placebo, which represents a 45% improvement over placebo when compared to the No Swim baseline.

[image]

An extract of FV was tested against placebo in a swim-to-exhaustion mouse study. The results below indicate that the FY extract increases stamina in the specificc test by over 5-fold.

[image]

### Example 4

An extract of FV was tested against placebo in a swim-to-exhaustion mouse study. After a 20 minute rest, the FV was shown to facilitate a surprising reduction in lactate. The placebo group showed no effective reduction after the same period.

[image]

### Example 6

An extract of FV was tested against 2nd Wind after a 10-minute swim study and a 20-minute recovery period. The FV improved lactic acid clearance 89% more than 2nd Wind.

[image]

1. A Flammulina velutipes extract, wherein the extract has the following characteristics: a moisture content ranging from 0 percent to 10 percent; a protein content ranging from 20 percent to 40 percent; a carbohydrate content ranging from 40 percent to 80 percent; an ash content ranging from 0 percent to 15 percent; and, a fat content ranging from 0 percent to 5 percent.

2. The extract according to claim 1, wherein the extract is made using a method comprising the following steps: comminution of a Flammulina velutipes material to form a powder; extraction of the powder using a solvent to provide a mixture of soluble and insoluble components; separating the soluble component from the insoluble component; concentrating the soluble component to provide a concentrate; and, drying the concentrate.

3. The extract according to claim 2, wherein the Flammulina velutipes material comprises one or more Flammulina velutipes fruiting bodies.

4. The extract according to claim 2, wherein the Flammulina velutipes material comprises fermented Flammulina velutipes mycelium.

5. The extract according to claim 2, wherein the Flammulina velutipes material comprises Flammulina velutipes fermentation medium comprising biochemical fermentation products.

6. The extract according to claim 2, wherein the Flammulina velutipes material consists essentially of one or more Flammulina velutipes fruiting bodies.

7. The extract according to claim 2, wherein the Flammulina velutipes material consists essentially of fermented Flammulina velutipes mycelium.

8. The extract according to claim 2, wherein the Flammulina velutipes material consists essentially of Flammulina velutipes fermentation medium comprising biochemical fermentation products.

9. The extract according to claim 2, wherein the Flammulina velutipes material consists essentially of Flammulina velutipes mycelium.

10. The extract according to claim 2, wherein the Flammulina velutipes material consists essentially of Flammulina velutipes spores.

11. A composition, wherein the composition comprises a Flammulina velutipes extract, and wherein the extract is formulated into a delivery form selected from a list consisting of capsules, tablets, a powdered drink mix, a ready-to-drink beverage, a semisolid, a food, and a supplement bar.

12. The composition according to claim 11, wherein the extract has the following characteristics: a moisture content ranging from 0 percent to 10 percent; a protein content ranging from 20 percent to 40 percent; a carbohydrate content ranging from 40 percent to 80 percent; an ash content ranging from 0 percent to 15 percent; and, a fat content ranging from 0 percent to 5 percent.

13. The composition according to claim 11, wherein the extract is formulated into a capsule, and wherein the formulated material further comprises at least one ingredient from the following list: flour; starch; modified starch; maltodextrin; cellulose; modified cellulose; protein hydrolysate; rice powder; whey powder; calcium phosphate; calcium carbonate; lactose; saccharides; sorbitol; mannitol; xylitol; stearic acid; stearate; silica; silicate, polyethylene glycol; flavors; and, colors.

14. The composition according to claim 11 7 wherein the extract is formulated into a tablet, and wherein the formulated material further comprises at least one ingredient from the following list: starch; modified starch; maltodextrin; cellulose; methylcellulose; ethylcellulose; hydroxypropylmethylcellulose; modified cellulose; protein hydrolysate; rice powder; whey powder; calcium phosphate; calcium carbonate; lactose; sweeteners; sorbitol; mannitol; xylitol; zein; saccharides; stearic acid; stearate; silica; silicate; polyethylene glycol; pharmaceutical glaze; wax; flavors; and colors.

15. The composition according to claim 1, wherein the extract is formulated into a powdered drink mix, and wherein the formulated material further comprises at least one ingredient from the following list: starch; modified starch; maltodextrin; cellulose; modified cellulose; protein hydrolysate; whey powder; calcium phosphate; calcium carbonate; lactose; sorbitol; mannitol; xylitol; sweeteners; stearic acid; stearate; silica; silicate; flavors; and, colors.

16. The composition according to claim 11, wherein the extract is formulated into a ready-to-drink beverage, and wherein the formulated material further comprises at least one ingredient from the following list: starch; modified starch; maltodextrin; cellulose; modified cellulose; protein hydrolysate; whey powder; calcium phosphate; calcium carbonate; lecithin; sweeteners; sorbitol; mannitol; xylitol; silica; silicate; solvents; acidifiers; citrate; preservatives; caffeine; flavors; and, colors.

17. The composition according to claim 11, wherein the extract is formulated into a semisolid, and wherein the formulated material further comprises at least one ingredient from the following list: starch; modified starch; maltodextrin; cellulose; modified cellulose; protein hydrolysate; whey powder; calcium phosphate; calcium carbonate; lecithin oil; partially hydrogenated oil; fat; milk; milk solids; mono- or diglycerides; polysorbates; sorbitan monostearate; sweeteners; sorbitol; mannitol; xylitol; silica; silicate; solvents; acidifiers; citrate; preservatives; flavors; and, colors.

18. The composition according to claim 11, wherein the extract is formulated into a food or supplement bar, and wherein the formulated material further comprises at least one ingredient from the following list: flour; starch; modified starch; maltodextrin; cellulose; methylcellulose; ethylcellulose; hydroxypropylmethylcellulose; modified cellulose; protein hydrolysate; whey powder; calcium phosphate; calcium carbonate; lecithin; mono- or diglycerides; polysorbates; sorbitan monostearate binders; sweeteners; sorbitol; mannitol; xylitol; silica; silicate; solvents; acidifiers; citrate; preservatives; flavors; and, colors.

19. A kit for improving lactic acid clearance, muscle reaction time, stamina, exertion recovery time, or reducing fatigue, wherein the kit comprises: a composition comprising a Flammulina velutipes extract; a container including the composition; and, instructions on how to use the composition to improve lactic acid clearance, muscle reaction time, stamina, exertion recovery time or to reduce fatigue.

20. The kit according to claim 19, wherein the composition is in the form of a capsule.

21. The kit according to claim 19, wherein the composition is in the form of a tablet.

22. The kit according to claim 19, wherein the composition is in the form of a powdered drink mix.

23. The kit according to claim 19, wherein the composition is in the form of a ready-to-drink beverage.

24. The kit according to claim 19, wherein the composition is in the form of a semisolid.

25. The kit according to claim 19, wherein the composition is in the form of a food or supplement bar.

26. A method for increasing physical stamina in a mammal, wherein the method comprises administering a composition comprising a Flammulina velutipes extract to the mammal.

27. The method according to claim 26, wherein the composition is in the form of a capsule.

28. The method according to claim 26, wherein the composition is in the form of a tablet.

29. The method according to claim 26, wherein the composition is in the form of a powdered drink mix that has been mixed with a solvent.

30. The method according to claim 26, wherein the composition is in the form of a ready-to-drink beverage.

31. The method according to claim 26, wherein the composition is in the form of a semisolid.

32. The method according to claim 26, wherein the composition is in the form of a food or supplement bar.
